(19) United States
(12) Patent Application Publication
(10) Pub. No.: US 2013/0018240 A1
(43) Pub. Date: Jan. 17, 2013

(54) BODY MEASUREMENT AND IMAGING WITH A MOBILE DEVICE

(71) Applicant: Kim McCoy, SAN DIEGO, CA (US)

(72) Inventor: Kim McCoy, SAN DIEGO, CA (US)

(21) Appl. No.: 13/448,186

(22) Filed: Apr. 16, 2012

Related U.S. Application Data
(60) Provisional application No. 61/507,100, filed on Jul. 12, 2011.
(60) Provisional application No. 61/545,080, filed on Oct. 7, 2011.

(51) Int. Cl. A61B 5/026; A61B 3/16; A61B 5/1455; A61B 5/00; H04M 1/00; G09B 23/28

(52) U.S. Cl. 600/323; 455/556.1; 434/262; 600/481; 600/407; 600/398

(57) ABSTRACT

A standard handheld cellular telephone for obtaining diagnostic data is described. In at least one aspect, a standard handheld cellular telephone has a processor, a cellular transceiver, a signal transmitter, a signal receiver and memory for storing instructions for execution by the processor. The acoustic, electromagnetic, optical, and positioning functions of a cellular telephone are exploited. The standard handheld cellular telephone is configured to be capable of performing operations including causing the signal transmitter to transmit a signal through a medium when the signal transmitter is placed adjacent to the medium, receiving with the signal receiver one or more propagations of the transmitted signal after the transmitted signal has passed at least in part through the medium, and obtaining diagnostic data from the received one or more signal propagations.

### CROSS-REFERENCE TO RELATED APPLICATIONS

This disclosure claims the benefit of U.S. Provisional Application No. 61/507,100, filed Jul. 12, 2011, and U.S. Provisional Application No. 61/545,080, filed Oct. 7, 2011, the disclosures of which are incorporated herein by reference in their entirety.

### BACKGROUND

Mobile devices such as cellular telephones and personal digital assistants (PDA) typically include various transmitters and receivers. For example, they may include microphones, voice encoders, decoders, power amplifiers, compasses, accelerometers, antennae, firmware, speakers, earpieces, camera, a light source, and the like.

### SUMMARY

This disclosure pertains to obtaining diagnostic data about a medium using a standard handheld cellular telephone. The merging of transducers, sensing technologies, and computing technologies provide new measurement techniques. For example, electromagnetic, optical, and acoustic functions of a mobile device can be individually or collectively exploited to obtain data (e.g., physiological measurement data) and provide imaging information from a medium using a mobile device not normally intended to be used for medical purposes.

In further detail, a handheld diagnostic device includes a standard handheld cellular telephone having a processor, a cellular transceiver, a signal transmitter, a signal receiver, and memory for storing instructions for execution by the processor. The standard handheld cellular telephone is configured to be capable of performing operations including causing the signal transmitter to transmit a signal through a medium when the signal transmitter is placed adjacent to the medium, receiving with the signal receiver one or more propagations of the transmitted signal after the transmitted signal has passed at least in part through the medium, and obtaining diagnostic data from the received one or more signal propagations.

Acoustics

Historically, medical acoustic measurements have utilized ultra-sound frequencies extending from 20 KHz to the 10s of MHz. Although existing techniques are useful in imaging at high frequencies these techniques do not excite or measure resonances (or propagation paths) at lower frequencies. A mobile device typically includes a telephone earpiece (e.g., a speaker) and a microphone that are used as an acoustic source and a receiver respectively. Such a source and receiver transmit and receive energy thus enabling data acquisition leading to, for example, the acoustic measurement and imaging of a medium. When acoustic energy from the acoustic source propagates through a medium, such as a portion of a human body, it takes many different paths. Each one of these ‘multipaths’ has a distinguishable path over which it dissipates its energy. The energy is uniformly radiated out along some paths while along others it is rapidly absorbed, refracted, or reflected. A signal's refraction path and the signal's absorption level are frequency dependent. For example, a signal propagated through diverse tissues along multiple paths of a human body part can include skin, muscle, vascular, organ, and skeletal effect changes; usually lower frequencies of the signal will propagate less disturbed through a medium and the higher frequencies will be more rapidly absorbed. Some frequencies will cause parts of the body, its structures, tissues, gas volumes, and fluids to resonate (including eye fluid, brain, etc.). The speed of sound varies as a function of tissue density; hence, each tissue type has its own energy propagation properties. The speed of sound in air is approximately 330 meters per second whereas the speed of sound in some body tissues is near the speed of sound in water, which is approximately 1500 meters per second. One or more acoustic receivers (microphones), including a built-in receiver within the mobile device, can sense the changes in energy propagation.

The mobile device can be programmed to exploit medical device techniques such as computed tomography (CT) or computed axial tomography (CAT) (e.g., where the patient is placed inside a large imaging instrument) to analyze the sensed changes in the energy propagation. The amount of energy arriving at a point is related to the distance between the transmitter and receiver and generally decays at the rate of 1/R², where R is equal to the distance between transmitter and receiver. When a mobile device such as a cellular telephone is used (instead of a large fixed device) the transmitter may be located at or near the surface of the body. This results in a significant decrease in an amount of energy that needs to be transmitted. It also results in a lower spatial uncertainty. Also, tomographic methods which exploit both time and frequency characteristics of a propagating signal (similar to those used in oceanography) can also be implemented in the mobile device to facilitate the analysis of the sensed changes in energy propagation.

For example, the mobile device can include a transmitter-receiver pair that can transmit an acoustic signal through a medium and sense reflected multipaths of the signals from the medium. For example, a transmitter-receiver pair can include an acoustic transmitter and an acoustic receiver that are collocated. In some examples, there can be an aperture (e.g., larger distance) between the transmitter receiver pair. Such an acoustic transmitter can transmit an acoustic signal and the acoustic receiver can sense a backscattered signal. When fixed over a known body part, physiological data, such as an acoustic image of the body, can be obtained as with an ultrasound examination for pre-natal care eliminating the need of a dedicated medical device. In some examples, the acoustic transmitter-receiver pair can be located at different locations on a medium. The acoustic receiver can sense multipaths from a signal transmitted through the medium.

Positioning

Also, a positioning system can be used to identify the location of a mobile device (or of a sensing device used by the mobile device) with respect to the medium. For example, the mobile device can use known reference points in a positioning system (e.g., ultra short base line (USBL) navigation using radio frequency identification (RFID) tags set up at known locations in a garment, on the body, or in a room) to image a medium such as a body part while being passed over that medium. The mobile device can identify its location by energizing RFID tags by providing a pulse of electromagnetic energy. This pulse of electromagnetic energy is used for positioning purposes and is separate from the acoustic pulse or waveform.

In some examples, an acoustic signal can be propagated through the chest of a patient. The transmitted signal can be sensed by the acoustic receiver of a mobile device. The spectral analysis can be performed on the received signal to identify resonance frequencies in the signal to determine the presence of a pneumothorax and other acoustic abnormalities. The spectral analysis can produce a spectrogram that can be compared with the spectrogram from a lung without a pneumothorax (e.g., the other lung of the patient without a pneumothorax). Such comparisons identify abnormal frequency responses to a pneumothorax or other acoustic abnormality of the body.

In some examples, an earphone-microphone jack can be used to connect a mobile device to a data collection device that includes an array of sources and/or receivers. The greater the number of source-receiver paths, the higher resolution suite of measurements that can be obtained. Multiple source-receiver locations (e.g., multiple geometries) allow higher resolution acoustic images of a medium to be obtained. Also, a data collection device that connects to an earphone-microphone jack can include a known aperture between a transmitter on the data collection device and a receiver on the data collection device. Such a data collection device can have multiple extensions with known apertures, so that measurements can be made at those different, known apertures.

Also, RFID tags fixed at known locations can also be equipped with acoustic receivers that receive an acoustic signal transmitted from a mobile device. The RFID tags can be used to identify a location of the mobile device when it transmits the acoustic signal. Also, when the transmitted signal is received at the respective receivers, the location of the received signals with respect to the mobile device can be identified because they are fixed at the known locations (i.e., with the RFID tags).

Electromagnetics and Bioelectromagnetism

The dielectric constant of a fluid is proportional to several of its physical and chemical properties including the bulk ion-concentration (i.e. cumulative specific) of the fluid. At a given temperature, the propagation of electromagnetic waves through a medium is proportional to its dielectric constant. The dielectric constant of a medium (e.g., a body part) is also frequency dependent. Electromagnetic signals (waveforms) composed of multiple frequencies and/or chirps can be transmitted through a medium to obtain data such as imaging information about that medium. For example, when a response function of the dielectric constant is viewed in the time-domain, a spatial image of the body can be obtained. The attenuation of a transmitted signal over a path is proportional to the dielectric properties along that path. Hence, the received signal is proportional to material of the medium (e.g., material of a body part) along the path the signal traveled. A signal transmitted into a medium such as a body part propagates along multiple paths. The multipath arrivals sensed by an electromagnetic receiver (e.g. antenna) provide information about the dielectric properties of each of those paths. Any variation in the bioelectromagnetism of a material or a foreign object within the path with a different dielectric constant will influence the received signal. The transmitted signal can be amplified, attenuated, or varied in frequency as needed to obtain data about the medium, such as to image the medium, through which the electromagnetic signal is passed.

A radio frequency identification (“RFID”) tag responds to a radio frequency (“RF”) signal and may be stimulated by an RF pulse initiated from a mobile device. Thus an RFID tag stimulated by the mobile device (such as a cellular telephone) will result in each of the RFID tags ‘responding’ with a finite amount of energy from its known location. An RFID tag implanted in or upon a medium such as a body will have a varied electromagnetic response function. Such variations are based upon the changes within the medium and the location(s) of the transmitter-receiver.

As an electromagnetic signal passes through a medium, changes in a signal phase, amplitude, and/or a frequency of the signal occur. Some frequencies will be attenuated and others may remain unchanged. These changes can be used to obtain data from the medium such as to image a medium. For example, the speed of propagation of radio waves through a vacuum is higher than through a fluid. If the frequency of a transmitted signal is known then the phase angle of the signal is also known as a function time. With the precise knowledge of time, any change in the phase angle of a signal is an indication of either physical movement or a change in the properties of the medium. Any the change of the dielectric properties across a measurement path will also change the received signal composition. Slower propagating signals will be retarded in phase.

Ionic fluids (such as blood and tissues) contain a diversity of ions each of different size, mass and electrical charge. When stimulated by an electromagnetic pulse an ion will align its polarity according to the stimulating electromagnetic field in accordance with its mass and charge. The alignment utilizes a finite amount of energy that is absorbed at or near a specific frequency. After the ion has aligned it will pass charge along the lines of the electromagnetic field. Each ion has a specific relaxation (and absorption) frequency at which it will can be excited and then resonate. Ion relaxation frequencies are typically in the megahertz to gigahertz ranges. A high absorption of energy at a specific frequency is proportional to a higher content of an ion with (or near) the transmitted frequency. The relaxation frequencies can be sensed and used to determine the ionic content of blood or tissue. Such bulk conductivity methods can be used to determine the moisture content of materials (e.g., hydration/dehydration). When a frequency sweeping signal is transmitted through a medium an energy absorption spectrum will identify the frequencies at which energy is being absorbed. As an RF signal passes through a medium, the ratio of ion mass to ion charge can be measured.

### DESCRIPTION OF DRAWINGS

FIG. 1 shows an example mobile device capable of transmitting acoustic and radio waves.

FIG. 2 shows an example of a mobile device being used to obtain data from a medium using transmission and reflective techniques.

FIG. 3 shows an example of components of a mobile device that can be used for obtaining data from a medium.

FIG. 4 shows a system for obtaining data from a medium.

FIG. 5-7 shows examples of acoustic transceiver devices.

FIG. 8 shows a side of a mobile device transmitting a signal into a medium and receiving the signal after it has been influenced by the medium.

FIG. 9 shows an example of the mobile device being used to obtain data from a medium using an electromagnetic transceiver.

FIG. 10 shows another example of obtaining data, such as imaging data, from a medium using multiple electromagnetic transceivers.

FIG. 11 shows clothing that includes multiple transceivers

FIG. 12 shows clothing that includes multiple radio frequency identification (RFID) tags

FIG. 13 shows a method for imaging a medium

FIG. 14 shows an example process for using a mobile device to detect a pneumothorax.

FIG. 15 shows an example spectrogram for a lung having a pneumothorax and an example spectrogram of a lung without a pneumothorax.

### DETAILED DESCRIPTION

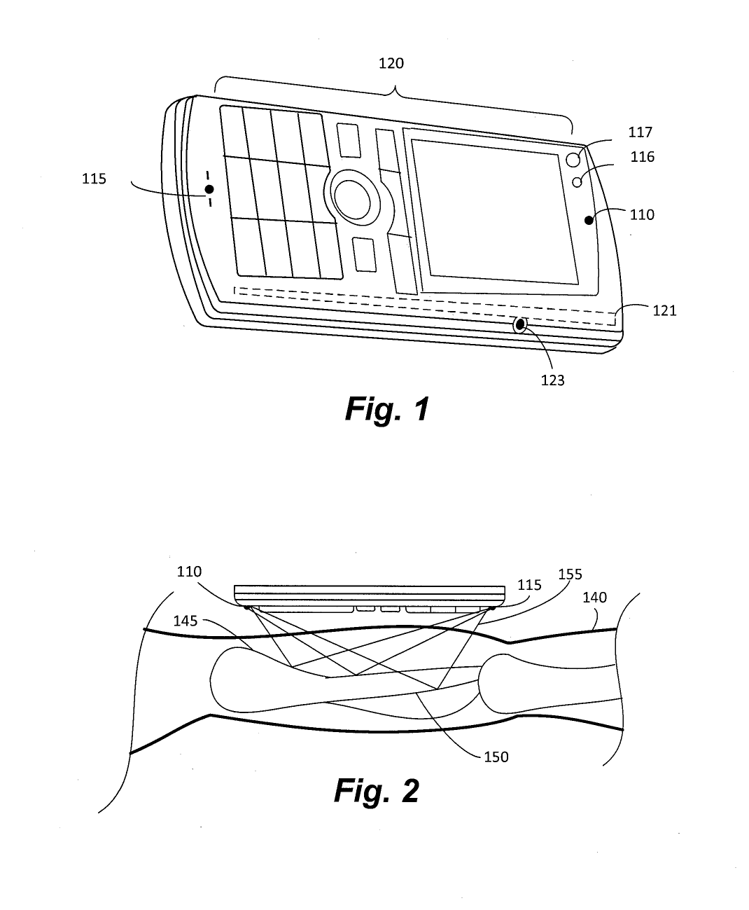

FIG. 1 shows an example mobile device 100 having an acoustic transmitter 110 and an acoustic receiver 115. The acoustic transmitter 110 can be a sound source such as a speaker or an ear piece for transmitting an acoustic signal. The acoustic receiver 115 can include a microphone. The acoustic transmitter and the acoustic receiver are separated by a distance referred to as an aperture 120. The acoustic transmitter 110 and the acoustic receiver 115 can be used, for example, during a mobile telephone call for communication between a user of the mobile device 100 and a remote user. Also, the acoustic transmitter 110 and the acoustic receiver 115 can be coupled to a medium to obtain an acoustic image of the medium.

The mobile device 100 also includes an electromagnetic transceiver 121, such as an antenna. The transceiver 121 is internal to the mobile device 100. The transceiver 121 can include a cellular transceiver for transmitting and receiving a cellular signal to facilitate cellular communication.

The mobile device 100 also includes an optical sensor or receiver 116 (e.g., a camera) and an optical source 117 (e.g. a light). The optical receiver 116 may receive spectral (color) information passively from a body part. The mobile device may also actively stimulate using, e.g., the optical source 117, and receive spectral information using, e.g., the optical receiver 116, from a body part to determine tissue health and oxygen saturation levels. The optical sensor 116 can capture an optical image such as a photograph or a series of optical images such as a video. The optical image(s) can be used in positioning the mobile device 100 with respect to a medium. For example, as a user positions the mobile device 100 with respect to a body part, the optical sensor can obtain an optical image of the body part. The mobile device 100 can process the image and identify the body part, and therefore the position of the mobile device 100 with respect to that body part.

FIG. 2 shows an example of the mobile device 100 being used to obtain data from a medium 140 using scattering and reflective techniques. The medium includes, within the medium, various surfaces, including a first surface 145 and a second surface 150. The mobile device including the transmitter 110 and the receiver 100 are coupled with the medium, such as by being pushed up against the medium 140. Signal propagation in unwanted directions may be abated if needed. The acoustic signal 155 produced by the transmitter 110 propagates through the medium 140 and reflects off of surfaces within the medium. Depending on the nature of the medium, including the density of the medium and the surfaces within the medium, the acoustic signal 155 travels along a set of pathways, referred to as multipaths. Along some of the pathways, the signal 155 is reflected back to the acoustic receiver 115. Because the speed of the signal 155 depends on the acoustic properties and density of the medium and because the pathways cover different distances, the signal received at the acoustic receiver along the different pathways arrives at different times, at different intensities with different signal compositions. The signal 155 can be used to identify characteristics of the medium including various surfaces within the medium 140.

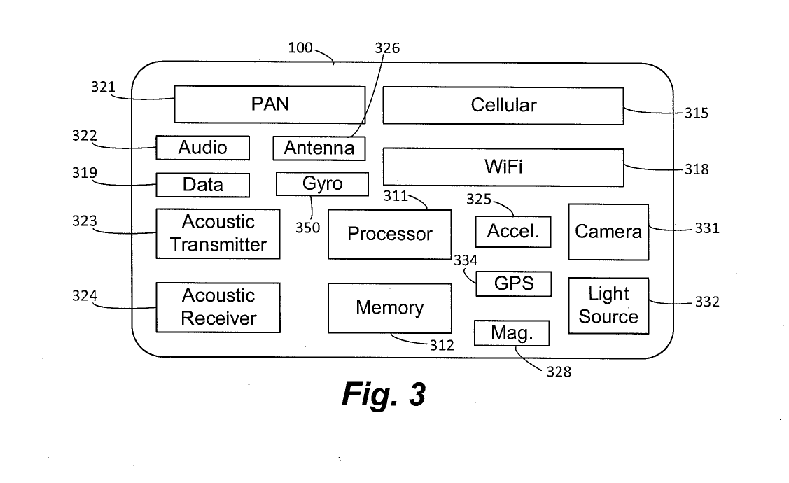

FIG. 3 shows an example of the mobile device 100 that can be used for obtaining data such as imaging data from a medium. The mobile device 100 can include a processor 311 and memory 312. The memory can store one or more programs with instructions that when run by the processor 311 cause the processor 311 to perform various functions, including obtaining data such as imaging data from a medium. The memory 312 can also store data obtained from the medium. The processor 312 can communicate with other components of the mobile device 100, including a cellular transceiver 315, Wi-Fi transceiver 318, a personal area network (“PAN”) transceiver 321 (e.g., Bluetooth™), audio input/output port 322, a data port 319, antenna port 326, acoustic transmitter 323, an acoustic receiver 324, an accelerometer 325, a magnetometer 328, a gyroscope 350 and a camera device 331 (configured to capture pictures and/or video), a light source 332, and a global positioning system (GPS) device 334. As discussed in more detail below, these various components can be controlled by the processor 311 in the mobile device 100 to obtain data such as image data from a medium.

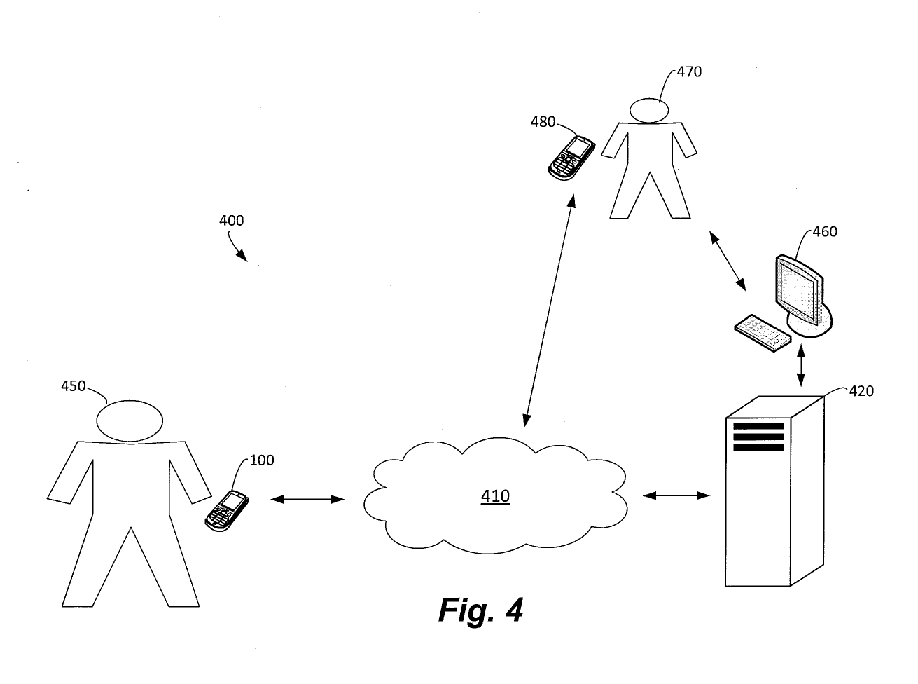

FIG. 4 shows an example system 400 for obtaining data such as imaging data from a medium. The system can include the mobile device 100 in use by a user 450, a network 410, and a computing apparatus 420 (e.g., a computer, a server, a server system, etc.). The mobile device 100 can be in two-way communication with the computing apparatus 420 over the network 410. The mobile device 100 can also provide data such as imaging data obtained at the mobile device 100 from the medium 140 to the computing apparatus 420 for further analysis. The imaging data can also be provided to a health care professional 470 (e.g., a doctor, a nurse, or a technician) for further review. The imaging data can be provided to the health care professional 470 directly from the computing apparatus 420 such as via a user interface devices 460 (e.g., a display device, and an input device). The imaging data can also be provided by the computing apparatus 420 or by the mobile device 100 over the network 410 to a computing device 480 (e.g., a handheld device) of the health care profession 470.

The mobile device 100 can receive instructions over the network 410 (and software residing in network) from the computing apparatus 420, such as instructions for imaging the medium 140. The instructions can include location instructions, directing the user 450 where and how to locate the mobile device 100 with respect to the medium 140. Also, the instructions can include instructions for the mobile device 100 regarding the nature and type of signal to be used in obtaining data such as imaging data from the medium 140. In some examples, when data is collected from the medium 140, the computing apparatus 420 and/or the health care professional 470, can analyze the data and provide additional instructions to the mobile device 100 regarding additional, different signals to be used in collecting data such as imaging the medium.

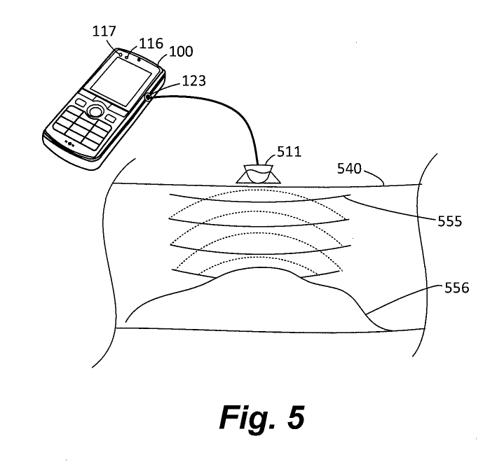

As shown in FIG. 1, the mobile device can also include audio input/output port 123 that can be connected with an external acoustic transceiver device. FIG. 5 shows an example of a data collection device that includes an acoustic transceiver device 511 connected to the mobile device 100 through the audio input/output port 123. In some examples, the acoustic transceiver device 511 can be connected to the mobile device through a wireless connection such as a through the PAN transceiver 321.

The transceiver device 511 includes, for example, a speaker that transmits an acoustic signal and sensor (e.g., a microphone) that can also receive an acoustic signal. As shown in FIG. 5, the transceiver device 511 has no aperture between transmitter and receiver because the transmitter and receiver are located at the same position. A user couples the transceiver device 511 to a medium 540. The mobile device 100 causes the transceiver device 511 to transmit an acoustic signal 555 (transmission shown by solid lines) into the medium 540. The transmitted acoustic signal reflects off of a surface 556 (reflected acoustic signal 555, shown by dashed lines). The reflected acoustic signal 555 can be received by the transceiver device 511 and used to image the surface 556 within the medium 540.

In some examples, the mobile device can be coupled to the medium 540 and transmit a signal into the medium using the acoustic transmitter 110 of the mobile device. The transceiver 511 can receive the signal. The distance between the transceiver and the mobile device 100 can be predetermined based on a known distance of a wire that couples the mobile device 100 and the transceiver 511. In some examples, various extensions of different known lengths can be used so that the distance between the mobile device 100 and the transceiver 511 can be measured. Also, the transceiver 511 can transmit a signal that is received by the acoustic receiver 115 of the mobile device 100.

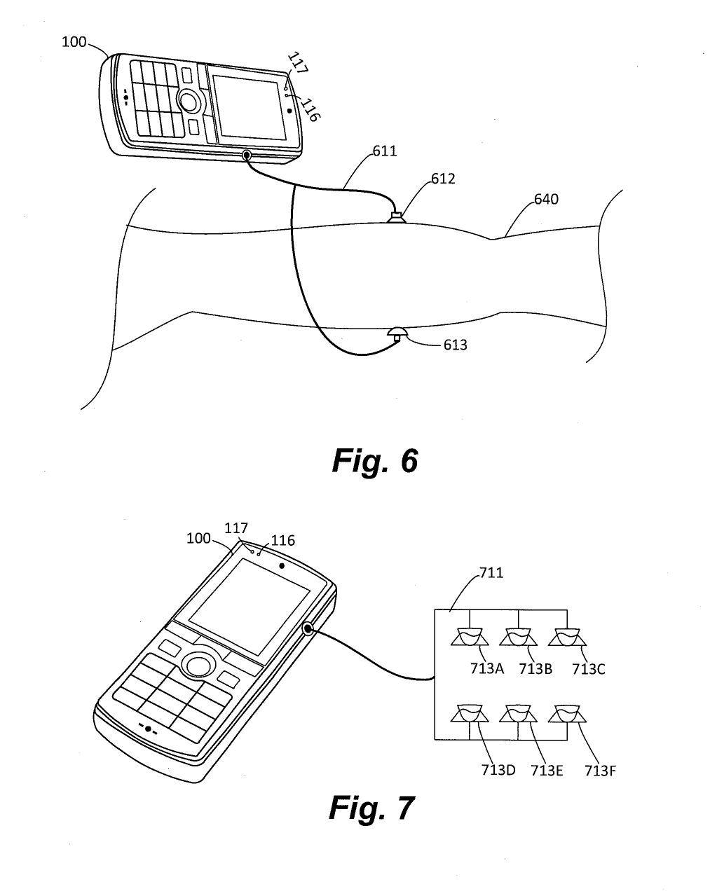

FIG. 6 shows another example of a data collection device in the form of an acoustic transceiver device 611 connected to the mobile device 100 through the audio input/output port 123. The transceiver device 611 includes an acoustic transmitter 612 and an acoustic receiver 613 (e.g., a microphone). The acoustic transmitter 612 can be placed on a side of a medium 640 and the acoustic receiver 613 can be placed on another side of the medium 640. The acoustic transmitter 611 can transmit an acoustic signal through the medium 640, which is received by the acoustic receiver 613. The acoustic signal, as it passes through the medium, will be altered based on the nature of the medium through which it propagates. The mobile device 100 can use the signal received at the receiver 613 to image the medium.

FIG. 7 shows an example of another data collection device in the form of an acoustic transceiver device 711 connected to the mobile device 100 through the audio input/output port 123. The transceiver device 711 includes multiple transceivers 713A-713F. Each of the transceivers 713A-713F includes an acoustic transmitter-receiver pair. The transceivers 713A-713F can be coupled with a medium at various different locations. In some examples, the distance between adjacent transceivers can be predetermined. Such location information can be used in imaging a medium.

One of the transceivers (e.g., 713A) can transmit a signal. The signal can propagate through the medium through various paths (i.e., mutlipaths), and can arrive at the other transceivers (e.g., 713B-F) altered according the characteristics of the path by which the signal traveled. The data obtained by the various other transceivers can be used to image a medium. For example, the acoustic energy along each of the paths can be analyzed as a function of time to determine properties of the medium along each of the respective paths. Because each of the multiple transceivers 712A-F can act as a transmitter and as a receiver, bidirectional imaging data can be obtained from the medium at each of the pairs. The imaging data from each of the multiple transceivers 712A-F can be analyzed by the mobile device 100. In some examples, the mobile device 100 can transmit the imaging data to a remote device over a network for further analysis and feedback.

In some examples, an electromagnetic transceiver can be used to obtain data from a medium, such as to image the medium. For example, an electromagnetic transceiver, such as the cellular transceiver 315, the Wi-Fi transceiver 3.18, and/or the personal area network (“PAN”) transceiver 3.21 (e.g., Bluetooth™) of the mobile device, can be used to obtain data such as image data from a medium. In some examples, an electromagnetic transceiver, coupled with the mobile device 100 through, for example, an antenna port, can be used to obtain data such as image data from the medium. Any electromagnetic signal of known composition may be received and used to obtain data such as image data, including the signal emanating from GPS satellites (e.g., L1 and L2). The frequencies of the received signal need to be within the receivable frequency bands of the receiving device. In some examples, the transmitter and the receiver are separate (i.e., the receiver need not initiate or transmit the signal).

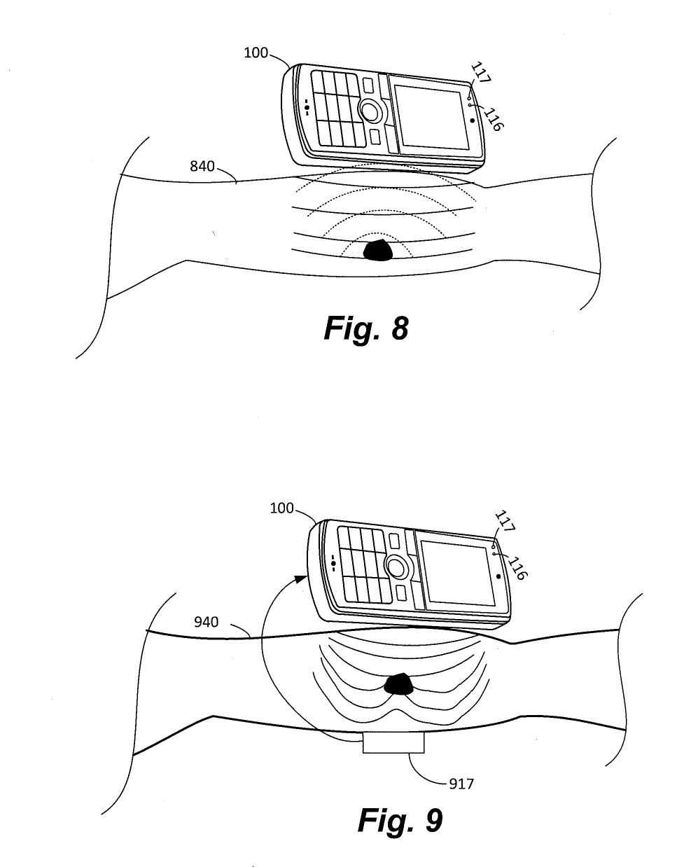

For example, based on the manufacturer specifications of the mobile device 100 and based on the type of measurement to be performed, the mobile device can provide instructions regarding how to couple the mobile device to a medium. For example, FIG. 8 shows a right side of the mobile device 100 coupled with a medium 840. A user of the mobile device 100 can be instructed to couple the right side of the mobile device 100 to the medium 840 because the electromagnetic transceiver 121 (e.g., cellular antenna) is on the right side of the mobile device 100. The transceiver can transmit an electromagnetic signal (e.g., a pulse) through medium 840 (shown in solid lines) and receive a backscatter of the signal (shown in dashed lines). The backscatter of the signal can be received at the electromagnetic transceiver.

In some examples, a cellular antenna in the mobile device 100 can be used to transmit a signal into the medium. Some cellular mobile devices include multi-band transceivers; a band of such a transceiver that is not in communication with a cell tower can be selected for use in obtaining data from a medium such as imaging data. Also, the band can be selected based on data collection needs (e.g., imaging needs). The signal transmitted by the cellular antenna can be sensed by the same cellular antenna as it returns in a backscatter. Such a backscatter can be used to image the medium 840. In some examples, a PAN transceiver can be programmed to transmit a signal into the medium 840 and can sense the backscatter from the signal. In some examples, a signal generated by a GPS device can be used to image a medium.

In some examples, a shield can be used (e.g., a metal cage) with the mobile device 100 to direct a signal emanating from the mobile device into the medium 840. In this manner, the signal emanating from the mobile device 100 can be omni-directional or quasi omni-directional. The shield can be included in the mobile device 100. In some examples, the shield can be externally coupled with mobile device. The mobile device can provide feedback to a user of the mobile device regarding how to position the mobile device with respect to the medium 840 and how to position the shield with respect to the mobile device. In some examples, an external antenna with a shield can be used to obtain data from the medium 840 and provide that data to the mobile device 100.

FIG. 9 shows an example of the mobile device 100 being used to obtain data such as image data from a medium 940 using an electromagnetic transceiver 917 external to the mobile device 100. The transceiver 917 can be connected with the mobile device 100, such as through the antenna port 326 or the data port 319. The external electromagnetic receiver 917 is coupled to a medium 940 and can receive an electromagnetic signal transmitted by the mobile device 100. The received signal at the external receiver 917 can be provided back to the mobile device 100 through, for example, the data port 319 or through the antenna port 326. In some examples, the external receiver 917 can be coupled to the mobile device 100 wirelessly such as by communicating with the PAN transceiver 321.

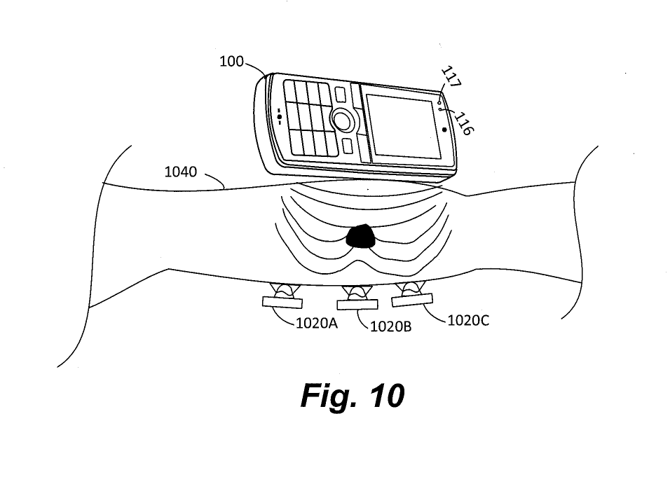

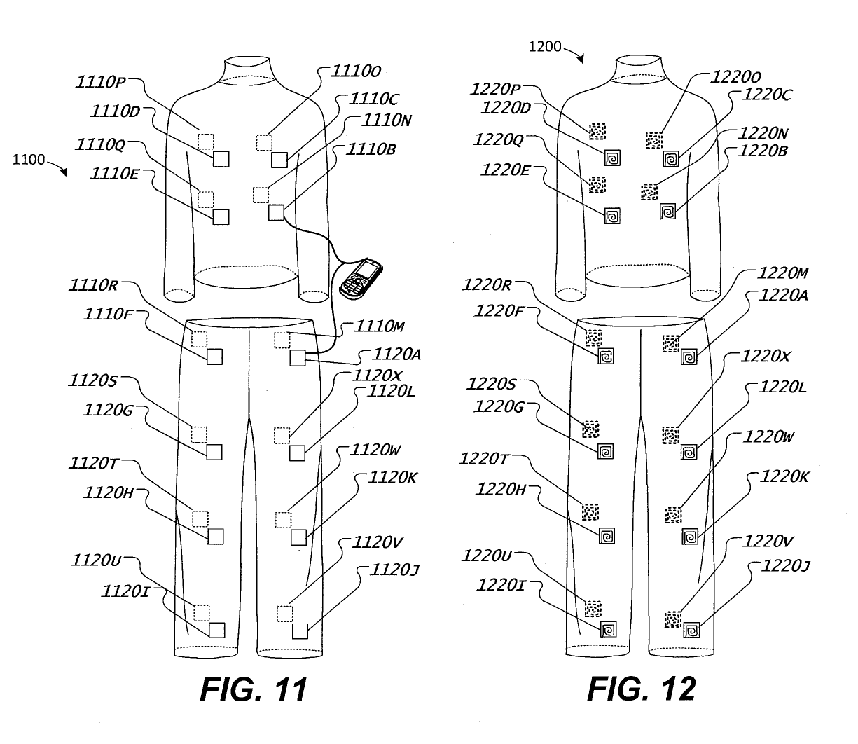

FIG. 10 shows another example of obtaining data such as imaging data from a medium 1040 using an electromagnetic transceiver in the mobile device 100. The mobile device 100 can transmit a signal through the medium 1040. One or more other electromagnetic transceivers 1020A-1020C can also be coupled with the medium 1040. The other transceivers 1020A-1020C can receive the signal transmitted by the mobile device 100 and can be programmed to repeat the received signal. The signal can be repeated after a predetermined time period. Each of the other transceivers 1020A-1020C can be programmed to transmit the signal at different times so as to not interfere with signals transmitted by each other. The mobile device 100 can receive the repeated signals from the other transceivers 1020A-1020C and can use the received signals to image the medium. In some examples, in response to receiving the signal from the mobile device 100, the other transceivers 1020A-1020C can be programmed to transmit a different signal back to the mobile device 100.

In some examples, the other sensors 1020A-1020C can be located with respect to each other and with respect to the medium 1040 using acoustical techniques. An acoustical pulse from the mobile device can be transmitted to sense the location of the transceivers 1020A-1020C. And, electromagnetic techniques as described above to image the medium.

The location of the other transceivers 1020A-1020C with respect to each other and with respect to the medium can be determined using the optical sensor 116. For example, the other transceivers 1020A-1020C can be placed in fixed locations with respect to the medium 1040. An optical image can be obtained using the mobile device 100 that shows the relative locations of the other transceivers. In some examples, one or more of the transceivers can have a known length, such as a dimension (i.e., a width of the transceiver) or an imprinted length that can be sensed by the optical sensor. The mobile device 100 can capture an optical image of the other transceivers 1020A-1020C. The length can be sensed from the captured optical image and used to determine the distance between the other transceivers 1020A-1020C.

Location information can provide important information in the obtaining of data from a medium such as imaging data. Location information can include location of a transmitter with respect to a receiver (e.g., the aperture 120 between the acoustic transmitter 110 and the acoustic receiver 115) and location of a transmitter and a receiver with respect to a medium being analyzed (e.g., imaged). By knowing location information the nature of multipaths of a signal transmitted through a medium can be used to map the medium more accurately. A computer program can be loaded onto the mobile device for imaging a medium. The computer program can obtain location information for the imaging of the medium. For example, the computer program can be obtain the aperture 120. The aperture 120 can be obtained from the device the manufacture or from a database that stores apertures by device make and model. Once the aperture is known, the signal 155 can be tailored to the aperture 120 and the medium 140 can be imaged.

FIG. 11 shows clothing 1100 that includes multiple transceiver 1120A-1120X. The transceivers 1120A-1120X can include, for example, multiple acoustic transceivers or can include multiple electromagnetic transceivers. By virtue of multiple transceivers 1120A-1120X location within the clothing 1100, the location of the multiple transceivers 1120A-1120X with respect to a body on which the clothing is worn is known. The transceivers 1120A-1120X can be connected with the mobile device 100. For example, as shown in FIG. 11, the transceivers can be connected to the mobile device 100 through a wired connection, such as through the data port 319, or through the audio port 322. In some examples, the multiple transceivers can be connected to the mobile device 100 wirelessly such as using the PAN transceiver 321.

If physiological data needs to be obtained regarding the heart (e.g., imaging data), for example, the transceiver 1120C can transmit a signal, such as an acoustic signal. The transceiver 1120C can receive data in the form of backscatter signals. Other of the multiple transceivers 1120B-X can also receive the signal as it travels through the body along various paths. The signal received by the multiple transceivers 1120A-1120X are stored by the mobile device 100 for imaging. The mobile device 100 can analyze the data and provide analysis results to the user. In some examples, the imaging data can be uploaded wirelessly to the remote computing apparatus 420 over the network 410. The remote computing apparatus can analyze the data and provide analysis results such as an image for the medium to the user 450 or to the health care professional 470 for further review. The remote computing apparatus can provide instructions to the mobile device 100. The instructions can include instructions regarding signal characteristics to be used to obtain data from the medium such as image data. Also, instructions can be provided to the user through the mobile device 100 regarding how or from what to collect data (e.g., image data).

In some examples, location information can also be obtained by the mobile device 100 to assist in the imaging of the medium. For example, a user can input information into the mobile device 100 indicating what medium the user is imaging. For example, if the user is imaging an arm, the user can input an indication into the mobile device that an arm is to be imaged, and can also input what part of the arm, e.g., the forearm. The mobile device can have a user interface that is activated by user input such as by voice, by touch input, by typing, or the like. The user can be presented with an application that helps the user identify what medium is to be imaged. For example, the user can indicate that the user wishes to image an arm. The user interface can present to the user a three dimensional image of an arm. The user can manipulate the three dimensional image to a location on the arm and can provide input indicating where the user wishes to obtain an image.

The mobile device 100 can also provide feedback or instructions regarding how and where to position the mobile device 100 or data collection device (e.g., data collection devices such as those discussed in FIGS. 5-10) connected to the mobile device to obtain a proper data such as image data. For example, if a user is using an acoustic transceiver device 611 as discussed in connection with FIG. 6, the mobile device can provide instructions to the user where to locate the acoustic transmitter and the acoustic receiver with respect to the medium (e.g., the arm). In this manner, the mobile device is provided location information. Also, once data such as imaging data is obtained, the mobile device 100 can provide instructions regarding moving the acoustic transceiver and/or the acoustic receiver (or other data collection device such as those discussed in FIGS. 1, and 5-10).

If the mobile device 100 itself is used to produce or receive an acoustic signal or an electromagnetic signal (as discussed in connection with FIGS. 1 and 8-10). The user can move the mobile device 100 from a starting point (such as a location from which first imaging data was obtained) to another position for obtaining additional image data such as instructed by the mobile device 100. In some examples, the accelerometer 325, gyroscope 350, and/or the magnetometer 328 in the mobile device 100 can provide feedback to the mobile device indicating a direction of movement and thereby providing an indication, relative to the starting position, from where the additional image data is being obtained.

In some examples, the location of the mobile device with respect to the medium can be obtained from an external device. For example, if the mobile device 100 is being use to transmit or receive a signal through the body of a subject wearing clothing that includes multiple acoustic transceivers 1120A-1120X as discussed in connection with FIG. 11, the location of the mobile device with respect to the body can be obtained by analyzing pings received from multiple of the various acoustic transceivers 1120A-1120X using, for example, ultra-short baseline monitoring techniques. Using time of flight based on a time base from the mobile device and triangulation, the location of the mobile device 100 with respect to those acoustic transceivers from which a signal was received can be determined. The mobile device can then use an electromagnetic signal and/or acoustic signal to, e.g., image the medium.

FIG. 12 shows clothing 1200 that includes multiple radio frequency identification (RFID) tags 1220A-1220X. Each of the RFID tags has a known location with respect to the other RFID tags and with respect to a body upon which clothing 1200 is worn. The mobile device can identify its location with respect to the body by transmitting an electromagnetic signal, such as a pulse, to activate the RFID tags. For example, if the mobile device 100 is held over the heart, an electromagnetic signal can be transmitted from the mobile device to activate RFID tag 1220N. The mobile device can detect a unique signal from the RFID tag 1220N and thereby locating the mobile device over the heart.

In some examples, the location of the mobile device 100 can be identified by external location devices. For example, a chair, a table, or a room where a user is located can be outfitted with location determining devices such as RFID tags, acoustic transmitters, etc. The location of the mobile device, including its movement, can be determined using the location determining devices on the chair, table, and/or room. For example, a user might enter a location such as a room that has multiple acoustic transmitters at fixed locations within the room. The acoustic transmitters can produce acoustic signals that are sensed by the mobile device 100, and using triangulation, the mobile device determines its location with respect to the room. As the mobile device 100 is moved, the change in location to a new location can be determined using the same technique. The acoustic transmitters can have a time based that is synced with the time base of the mobile device 100 to assist in the positioning of the mobile device 100 and any analysis done using the mobile device 100. For example, a common cell phone time base can be used. In some examples, a reference GPS time base can be used. In some examples, a time base can be transmitted across an electromagnetic signal to assist in the acoustic analysis (e.g., acoustic imaging) of a medium.

In some examples, the mobile device 100 can send out an acoustic signal which is received by the acoustic transmitters at fixed locations within the room. In response to receiving the signal from the mobile device 100, the acoustic transmitters produce a response signal. The mobile device 100 detects the response signal. The time from the transmission of the initial acoustic signal until the response is detected is calculated by mobile device. The calculated time can be used to measure the distance from the mobile device to the acoustic transmitters at fixed locations within the room. In this manner, the location and movement of the mobile device can be detected.

In some examples, the mobile device can determine that it is in a location outfitted with location determining devices based on a GPS signal. For example, a database stored by the mobile device 100 or by the computing apparatus 420 can have a list of locations with location determining devices. When a user indicates to the mobile device 100 that the user is going to obtain data from a medium (e.g., image the medium), the mobile device 100 can use a current GPS reading from the GPS device 334 on the mobile device 100 to check the database to determine whether the mobile device 100 is within such a location with such external location determining devices. If so, the mobile device 100 can obtain information about the external location determining devices such as from the database, including their location with respect to each other (e.g., where such location determining devices are located within a room). The mobile device 100 can use the external location determining devices to monitor its movement while the mobile device 100 is used to obtain data from the medium (e.g., image the medium).

In some examples, the multiple transceivers 1120A-1120X shown in FIG. 11 can include the RFID tags as shown in FIG. 1200. The RFID tags 1220A-1220L can be co-located with the multiple transceivers 1120A-1120X. For example, when an acoustic image is being taken, the RFID tags can be used identify a location of a signal transmitted by the mobile device 100 and can also provide location information regarding the received signal at the various multiple transceivers 1120A-1120X.

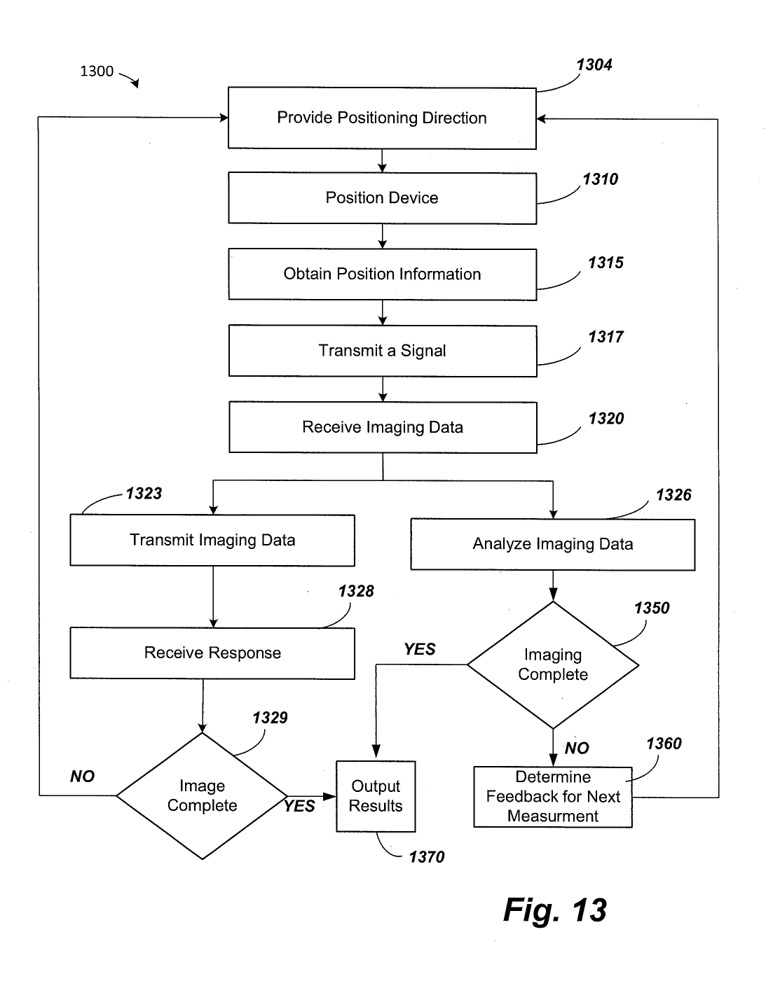

FIG. 13 shows a method 1300 for obtaining and analyzing data from a medium. For example, the method 1300 is shown in the context of imaging a medium. At 1304, a mobile device can provide positioning directions to the user. For example, if a user wants to perform imaging of the heart, the mobile device can provide instructions to the user regarding how and/or where to position the mobile device (or other data collection device connected with the mobile device) on the body being imaged. At 1310, a user of the mobile device positions the mobile device (or other data collection device connected with the mobile device) as instructed. At 1315, position information is obtained. For example, a positioning system such as from an RFID tag or using acoustic triangulation system (as described above) can be used to determine the position of the mobile device (or other data collection device connected with the mobile device). At 1317, a signal is transmitted from the mobile device into the medium. Positioning information can also be obtained from an optical sensor such as a camera. For example, an photograph or photographs (e.g., video) can be taken of the medium; the photograph or photographs can used by the mobile device or by a remote computing apparatus to identify the medium and/or to identify the location of the mobile device with respect to the medium. A photograph or photographs of the data collection devices can be obtained to identify the location of the collection devices with respect to each other, with respect to the medium, and/or with respect to mobile device.

Imaging data is received at 1320 as a result of the transmitted signal at the mobile device. For example, if an acoustic signal is transmitted, data over multiple acoustic paths can be obtained. If an electromagnetic signal is transmitted, data from multiple electromagnetic paths can be obtained.

In some examples, the mobile device uses the imaging data obtained by the mobile device to produce imaging results at the mobile device itself. For example, at 1326, image data is analyzed at the mobile device. If the imaging is not complete, feedback for a next measurement is determined at 1360. At 1304, new positioning directions are provided to the user regarding how and/or where to reposition the mobile device (or the other imagining device connected with the mobile device). The user repositions the mobile device (or other imagining device connected with the mobile device) as instructed at 1310. Position information can be obtained at 1315. In some examples, the positioning information can be obtained from a positioning system. In some examples, the positioning information can be obtained from the mobile device. For example, the mobile device can sense movement and direction of movement of the mobile device using an accelerometer in the mobile device. In this manner, a relative location with respect to a starting position (i.e., the position of the first measurement) can be obtained. At 1317, another signal is transmitted at 1317 into the medium. And, at 1320, imaging data can be received. Steps 1304-1326 can be repeated multiple times until it is determined that imaging is complete at 1350. In some instances, the feedback determined at 1360 can include signal characteristics of the signal to be transmitted at 1317 (e.g., amplitude, frequency, wavelength, shape of the signal, ramping, monotone, multiple tones etc.). When the imaging is complete, the results can be output for review by the user, for transmission to a remote health care professional, or output for further processing.

In some examples, the mobile device can transmit the imaging data to a remote server system for analysis at 1323. The remote server system can provide a response to the mobile device indicating that imaging is not complete and further imaging data is to be obtained. If imaging is not complete, steps 1304-1328 can be repeated. In some examples, the response received from the remote server system at 1328 can include further instructions regarding how to obtain imaging data. For example, the remote server system can provide instructions regarding how to position or use the mobile device (or other data collection device connected with the mobile device). In some examples, the remote server system can provide data regarding the characteristics of the signal to be transmitted at 1317.

At 1323, imaging data can also be provided to a health care professional for further review. The health care professional can provide feedback, including new positioning information, new signal characteristics, etc., to the remote server system for inclusion in the response. When the imaging is complete 1329, the results can be output 1370 to the health care professional, to the remote server system, and/or to the user.

The systems and techniques described herein can be used for multiple different applications, many of which include imaging a human subject. For example, the systems and techniques described herein can be used to determine the size and type of object within the human body. For example, organs can be imaged to determine the size of the organs. Foreign objects within the human body can be identified, such as a bullet or shrapnel using such techniques. For example, a soldier can be equipped with an underclothing with multiple transceivers (e.g., acoustic, electromagnetic, or both) as shown in FIG. 12. When the soldier is injured in the battle field, a mobile device can be connected to the multiple transceivers to imaging various parts of the body. The imaging results can identify foreign objects within the body of the soldier. This data can be transmitted by the mobile device to a remote server system for further analysis or provided to a remote health care professional for review.

In some examples, the systems and techniques can be used to image blood flow, pressure, and vascular status. For example, using an acoustic transmitter and receiver, such as shown in FIG. 6, an acoustic signal can be transmitted along a direction of blood flow at a predetermined tone. Blood flow can be imaged by measuring a Doppler shift in the frequency of the signal. Doppler shift measurements can be used to analyze heart hemodynamics. In some examples, a transmitter/receiver pair can be placed on a chest and back of a human subject for receiving acoustic imaging data to analyze vascular conditions of the human subject such as when the human subject is exercising.

In some implementations, the systems and techniques can be used to measure ocular pressures. Ocular pressures, geometries and anomalies intraocular pressure (typically between 10 and 20 mmHg) can be measured using non-contact methods. Proper positioning on the body can be established using the camera and accelerometer of the device. The eye is slightly deformed when the device emits sound pressure waves. Eye deformations (or anomalies) can be measured by the acoustic wave reflections, resonances, and phase changes as a function of the intraocular pressures and geometries. Optically, during eye deformation, the spectral content of light is influenced by the angles and the reflected return distance along the cornea-lens-retina path.

In some examples, bubble sizes in blood can be measured with the systems and techniques described herein. For example, the resonance frequency of a bubble is proportional to its diameter and signal loss is proportional to the ‘bubble fraction’. Using such methods both the amount of bubble in a fluid and the velocity of the bubbles can be determined.

For example, the systems and techniques described herein can be used to detect a pneumothorax (e.g., fully or partially collapsed lung). In a normal, healthy person the parietal and visceral pleura are in close proximity and separated by the parietal cavity fluid. When a gas (e.g., air) enters the pleural cavity the condition is referred to as pneumothorax. Smaller pneumothoraces may be asymptomatic and difficult to diagnose even by a trained medical professional without the aid of a chest x-ray.

A healthy lung is mostly composed of small volumes of air and surrounding tissue. Acoustic energy propagating through a body will cause abnormal internal gas volumes to resonate when compared to normal body tissues and fluids. The size of a pneumothorax, for example, determines the resonance frequency of the pneumothorax. Larger pneumothoraxes can produce lower resonance frequencies. For gases at a given pressure, resonant frequencies are inversely proportional to the resonating volume.

The acoustic transmitter 110 of the mobile device 100 and an acoustic receiver 115 (or an external acoustic transceiver device) can be used as an acoustic probe to transmit an acoustic signal into a body cavity and receive the acoustic signal after it has passed through the body cavity. For example, the pleural cavity area of the body can be ensonified. The mobile device 100 can identify resonances in the received acoustic signal from the pleural cavity. The received signal is analyzed using spectral techniques such as a Fast Fourier Transform (FFT) or may use methods exploiting changes in phase or intensity.

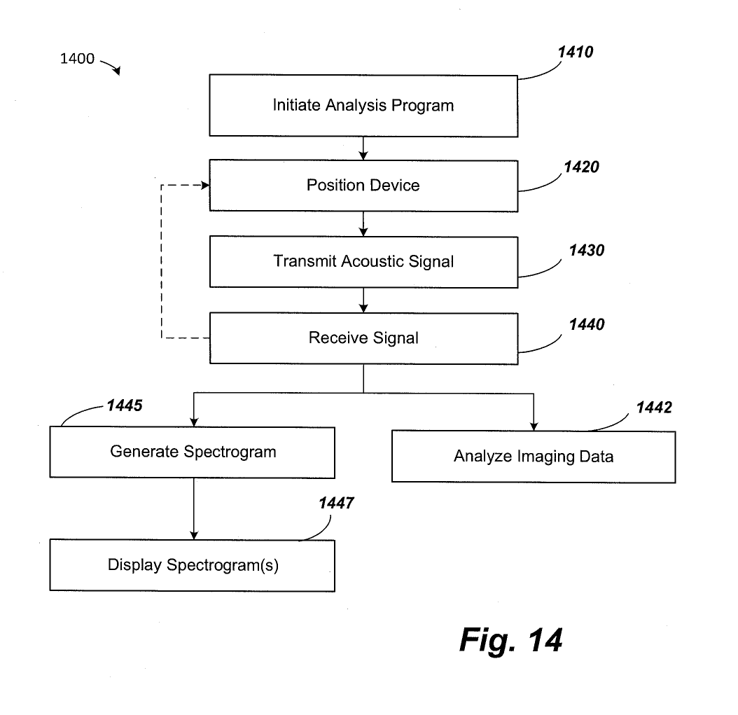

FIG. 14 shows an example process 1400 for using a mobile device, e.g., mobile device 100, to detect a pneumothorax. At 1410, a program for recording and/or analyzing an acoustic signal is initiated on the mobile device. At 1420, the mobile device is positioned with a receiver over a lung, such as on the chest of a patient. At 1430, an acoustic signal is generated and transmitted into the body passing through the pleural cavity and the lung. The acoustical signal can be generated (e.g., using the program) by the mobile device or a transceiver attached to the mobile device. In some examples, the signal is generated by tapping the chest (or back) such as with two fingers or with a rubber hammer. The tapping can be repeated multiple times to increase the accuracy of pneumothorax detection.

At 1440, the signal can then be received by an acoustic sensor (e.g., an acoustic transceiver) after it has propagated through at least a portion of the patient. At 1442, the return signal can then be analyzed using spectral analysis to identify the existence of resonance frequencies. The existence of resonance frequencies can be indicators of a potential pneumothorax.

In some examples, if the patient's other lung is healthy (e.g., known to be without a pneumothorax), steps 1420-1440 can be repeated with the patient's other lung. The results from the other lung can be compared against those of the lung being analyzed to identify abnormal resonances indicative of a pneumothorax or a partial (small) pneumothorax. A program on the mobile device or on a remote device (e.g., that of a health care professional connected to the mobile device over a network) can perform such an analysis.

In some examples, at 1445 the mobile device can generate for display a spectrogram for both lungs. The spectrogram is displayed at 1447 on the mobile device or transmitted for display to a remote device such as that of a health care professional for further analysis.

Process 1400 can be combined with the systems and techniques described herein to detect a pneumothorax. For example, the process 1400 can be performed using the systems described in connection with FIGS. 1-12. Also, process 1300 can be used to detect a pneumothorax to properly position the mobile device with respect to the lung. Also, the patient and/or the mobile device can be given further instructions from a remote computing device (e.g., over a network) for obtaining and computing spectral data from a patient to test for a pneumothorax.

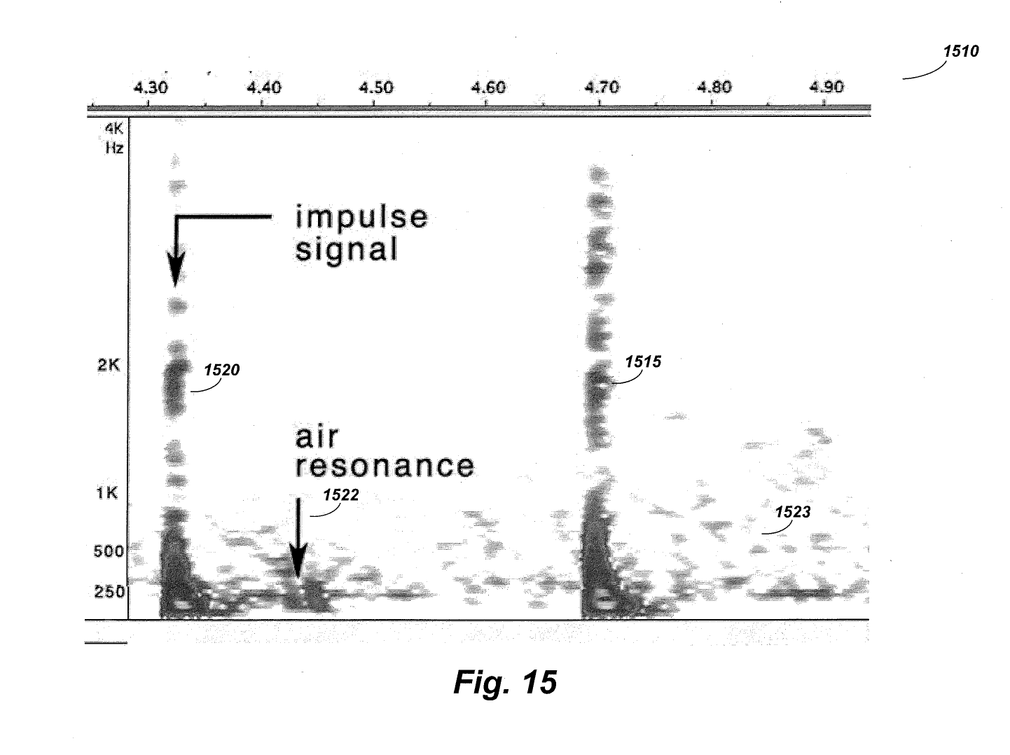

FIG. 15 shows an example spectrogram 1510 for a lung having a pneumothorax (left) and for a lung without a pneumothorax (right). The x-axis represents time and the y-axis represents frequency. In the spectrogram 1510, a propagation of a first signal 1520 (e.g., in the form of a ‘tap’) occurs at 4.32 seconds followed by a resonant return 1522 after ˜0.100 seconds centered around ˜250 Hz representative of at least a partial pneumothorax (an acoustic abnormality). The spectrogram 1510 shows a second signal 1515 (e.g., for the right lung) with the acoustic energy resonant returning randomly distributed 1523 up to ˜1000 Hz, representative of a lung without a large cavity resonance. The spectrogram 1510 exhibits low frequency resonance for the first signal 1520 and a more broadband series of small resonances extending up to 1000 Hz for the second signal 1520. The elapsed time between the transmission and the reception of signal is an indicator of distance and can aid in the location of anomalies.

In some examples, the propagating signal can be made of a series of short pulses extending from approximately 30 Hz to 8000 Hz to produce a clearer set of spectral images. Lower frequencies can be used to detect resonances for larger pneumothoraxes and higher frequencies can be used to detect resonances for smaller pneumothoraxes. Other lung disorders such as emphysema can also exhibit resonances and spectral characteristics depending on size, location, and distribution. Such lung disorders can also be detected with the systems and techniques described herein.

According to the techniques described above, a mobile device can have a spectral analysis program that provides much better resolution and accuracy in detecting a pneumothorax than the human ear. Such a technique can allow for the testing of a pneumothorax without needing to visit a doctor's office.

Also, an electromagnetic transmitter and receiver can also be used to detect a bubble in blood. Because the bulk conductivity of blood is different from the bulk conductivity of a bubble, an electromagnetic signal passed through the blood is affect by the change in bulk conductivity. By detecting the change of an electromagnetic signal as it passes along a path in a medium that includes blood and bubbles, the existence of bubbles can be detected.

As a signal propagates through an inhomogeneous medium the signal timing and composition changes. When the individual path of a signal is known, information about changes in the medium along that path can be determined. In some examples, by imaging surfaces and objects within a medium irregularities (perturbations to a prior condition), such as broken bones or hematomas can be identified. Also, bone density measurements can be obtained. For example, using an electromagnetic transceiver, dielectric properties of a bone within a medium can be imaged. Using the dielectric properties, bone density can be measured.

In some examples, bulk conductivity can be measured to identify contents of a medium. For example, an electromagnetic signal can be transmitted into a medium such as a body part with blood. Because different molecules absorb electromagnetic energy differently, content of the blood or body part can be analyzed with a signal that has a sweeping frequency. The signal as sensed can indicate, for example, the ionic content of blood because each ion has a specific relaxation frequency. The relaxation frequencies can be sensed and used to determine the ionic content of blood. Also, dehydration of tissue can be measured since the ionic concentration is increased during dehydration and increased during hydration.

The operations described in this specification can be implemented as operations performed by a data processing apparatus on data stored on one or more computer-readable storage devices or received from other sources. The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array), an ASIC (application-specific integrated circuit) or System On a Chip (SOC). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus.

A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices such as on or associated with a server or other computing device).

While this specification contains many specific implementation details, these should not be construed as limitations on the scope of any implementations or of what may be claimed, but rather as descriptions of features specific to particular implementations of particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

While operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations. Accordingly, other implementations are within the scope of the following claims.

What is claimed is:

1. A handheld diagnostic device comprising:
a standard handheld cellular telephone having a processor, a cellular transceiver, a signal transmitter, a signal receiver, and memory for storing instructions for execution by the processor; and
wherein the standard handheld cellular telephone is configured to be capable of performing operations including:
(a) causing the signal transmitter to transmit a signal through a medium when the signal transmitter is placed adjacent to the medium;
(b) receiving with the signal receiver one or more propagations of the transmitted signal after the transmitted signal has passed at least in part through the medium; and
(c) obtaining diagnostic data from the received one or more signal propagations.

2. The handheld diagnostic device of claim 1, wherein the signal transmitter comprises an acoustic speaker, and the signal receiver comprises an acoustic microphone.

3. The handheld diagnostic device of claim 2, wherein:
causing the signal transmitter to transmit a signal through a medium comprises causing the acoustic speaker to transmit an acoustic signal at a predetermined tone along a direction of blood flow in a body part; and
obtaining diagnostic data from the received one or more signal propagations comprises measuring a Doppler shift in a frequency of the transmitted signal and the one or more signal propagations, wherein the diagnostic data includes data regarding at least one of blood flow, vascular status, or heart hemodynamics.

4. The handheld diagnostic device of claim 1, wherein the signal transmitter comprises a light emitter, and the signal receiver comprises an image sensor.

5. The handheld diagnostic device of claim 4, wherein:
causing the signal transmitter to transmit a signal through a medium comprises causing the light emitter to transmit light through a body part;
receiving with the signal receiver one or more propagations of the transmitted signal comprises receiving with the image sensor spectral information after the transmitted light has passed at least in part through the body part; and
obtaining diagnostic data from the received one or more propagations comprises obtaining data regarding tissue health or oxygen saturation levels.

6. The handheld diagnostic device of claim 1, wherein the signal transmitter comprises an electromagnetic transmitter, and the signal receiver comprises an electromagnetic receiver.

7. The handheld diagnostic device of claim 6, wherein:
causing the signal transmitter to transmit a signal through a medium comprises causing the electromagnetic transmitter to transmit an electromagnetic signal through a body part; and
obtaining diagnostic data from the received one or more signal propagations comprises detecting a change between the electromagnetic signal and the received one or more signal propagations, wherein the diagnostic data includes data regarding at least one of bulk conductivity of the body part, bulk conductivity of blood in the body part, specific ionic content of blood in the body part, or dehydration of tissue in the body part.

8. The handheld diagnostic device of claim 6, wherein:
causing the signal transmitter to transmit a signal through a medium comprises causing the electromagnetic transmitter to transmit an electromagnetic signal to excite a radio frequency (RF) transmitter at a known location on or in a body part; and
receiving with the signal receiver one or more propagations of the transmitted signal comprises receiving an excitation signal from the RF transmitter.

9. The handheld diagnostic device of claim 1, wherein the medium comprises a body part, and the diagnostic data comprises data regarding the size of an organ in the body part.

10. The handheld diagnostic device of claim 1, wherein the medium comprises a body part including bone, and the diagnostic data comprises data regarding bone density.

11. The handheld diagnostic device of claim 1, wherein the medium comprises a body part including an eye, and the diagnostic data comprises data regarding at least one of ocular pressure, eye geometry, or eye anomalies.

12. The handheld diagnostic device of claim 1, wherein the medium comprises a body part, and the diagnostic data comprises data regarding a foreign object in the body part.

13. The handheld diagnostic device of claim 1, wherein the standard handheld cellular telephone is configured to be capable of performing operations further including:
providing instructions regarding how to position the standard handheld cellular telephone at a location adjacent to the medium;
performing operations (a), (b), and (c);
providing instructions regarding how to reposition the standard handheld cellular telephone to a new location adjacent to the medium; and
repeating the operations (a), (b), and (c).

14. A method of manufacturing a handheld diagnostic device, the method comprising:
obtaining a standard handheld cellular telephone having a processor, a cellular transceiver, a signal transmitter, a signal receiver, and memory for storing instructions for execution by the processor; and
configuring the standard handheld cellular telephone to be capable of performing operations including:
(a) causing the signal transmitter to transmit a signal through a medium when the signal transmitter is placed adjacent to the medium;
(b) receiving with the signal receiver one or more propagations of the transmitted signal after the transmitted signal has passed at least in part through the medium; and
(c) obtaining diagnostic data from the received one or more signal propagations.

15. The method of claim 14, wherein the signal transmitter comprises an acoustic speaker, and the signal receiver comprises an acoustic microphone.

16. The method of claim 15, wherein:
causing the signal transmitter to transmit a signal through a medium comprises causing the acoustic speaker to transmit an acoustic signal at a predetermined tone along a direction of blood flow in a body part; and
obtaining diagnostic data from the received one or more signal propagations comprises measuring a Doppler shift in a frequency of the transmitted signal and the one or more signal propagations, wherein the diagnostic data includes data regarding at least one of blood flow, vascular status, or heart hemodynamics.

17. The method of claim 14, wherein the signal transmitter comprises a light emitter, and the signal receiver comprises an image sensor.

18. The method of claim 17, wherein:
causing the signal transmitter to transmit a signal through a medium comprises causing the light emitter to transmit light through a body part;
receiving with the signal receiver one or more propagations of the transmitted signal comprises receiving with the image sensor spectral information after the transmitted light has passed at least in part through the body part; and
obtaining diagnostic data from the received one or more propagations comprises obtaining data regarding tissue health or oxygen saturation levels.

19. The method of claim 14, wherein the signal transmitter comprises an electromagnetic transmitter and the signal receiver comprises an electromagnetic receiver.

20. The method of claim 19, wherein:
causing the signal transmitter to transmit a signal through a medium comprises causing the electromagnetic transmitter to transmit an electromagnetic signal through a body part; and
obtaining diagnostic data from the received one or more signal propagations comprises detecting a change between the electromagnetic signal and the received one or more signal propagations, wherein the diagnostic data includes data regarding at least one of bulk conductivity of the body part, bulk conductivity of blood in the body part, specific ionic content of blood in the body part, or dehydration of tissue in the body part.

21. The method of claim 19, wherein:
causing the signal transmitter to transmit a signal through a medium comprises causing the electromagnetic transmitter to transmit an electromagnetic signal to excite a radio frequency (RF) transmitter at a known location on or in a body part; and
receiving with the signal receiver one or more propagations of the transmitted signal comprises receiving an excitation signal from the RF transmitter.

22. The method of claim 14, wherein the medium comprises a body part, and the diagnostic data comprises data regarding the size of an organ in the body part.

23. The method of claim 14, wherein the medium comprises a body part including bone, and the diagnostic data comprises data regarding bone density.

24. The method of claim 14, wherein the medium comprises a body part including an eye, and the diagnostic data comprises data regarding at least one of ocular pressure, eye geometry, or eye anomalies.

25. The method of claim 14, wherein the medium comprises a body part, and the diagnostic data comprises data regarding a foreign object in the body part.

26. The method of claim 14, wherein the standard handheld cellular telephone is configured to be capable of performing operations further including:
providing instructions regarding how to position the standard handheld cellular telephone at a location adjacent to the medium;
performing operations (a), (b), and (c);
providing instructions regarding how to reposition the standard handheld cellular telephone to a new location adjacent to the medium; and
repeating the operations (a), (b), and (c).

27. A method performed by a standard handheld cellular telephone having a processor, a cellular transceiver, a signal transmitter, a signal receiver, and memory for storing instructions for execution by the processor, the method comprising:
causing the signal transmitter to transmit a signal through a medium when the signal transmitter is placed adjacent to the medium;
receiving with the signal receiver one or more propagations of the transmitted signal after the transmitted signal has passed at least in part through the medium; and
obtaining diagnostic data from the received one or more signal propagations.

28. The method of claim 27, wherein the signal transmitter comprises an acoustic speaker, and the signal receiver comprises an acoustic microphone.

29. The method of claim 28, wherein:
causing the signal transmitter to transmit a signal through a medium comprises causing the acoustic speaker to transmit an acoustic signal at a predetermined tone along a direction of blood flow in a body part; and
obtaining diagnostic data from the received one or more signal propagations comprises measuring a Doppler shift in a frequency of the transmitted signal and the one or more signal propagations, wherein the diagnostic data includes data regarding at least one of blood flow, vascular status, or heart hemodynamics.

30. The method of claim 27, wherein the signal transmitter comprises a light emitter, and the signal receiver comprises an image sensor.

31. The method of claim 30, wherein:
causing the signal transmitter to transmit a signal through a medium comprises causing the light emitter to transmit light through a body part;
receiving with the signal receiver one or more propagations of the transmitted signal comprises receiving with the image sensor spectral information after the transmitted light has passed at least in part through the body part; and
obtaining diagnostic data from the received one or more propagations comprises obtaining data regarding tissue health or oxygen saturation levels.

32. The method of claim 27, wherein the signal transmitter comprises an electromagnetic transmitter, and the signal receiver comprises an electromagnetic receiver.

33. The method of claim 32, wherein:
causing the signal transmitter to transmit a signal through a medium comprises causing the electromagnetic transmitter to transmit an electromagnetic signal through a body part; and
obtaining diagnostic data from the received one or more signal propagations comprises detecting a change between the electromagnetic signal and the received one or more signal propagations, wherein the diagnostic data includes data regarding at least one of bulk conductivity of the body part, bulk conductivity of blood in the body part, specific ionic content of blood in the body part, or dehydration of tissue in the body part.

34. The method of claim 32, wherein:
causing the signal transmitter to transmit a signal through a medium comprises causing the electromagnetic transmitter to transmit an electromagnetic signal to excite a radio frequency (RF) transmitter at a known location on or in a body part; and
receiving with the signal receiver one or more propagations of the transmitted signal comprises receiving an excitation signal from the RF transmitter.

35. The method of claim 27, wherein the medium comprises a body part, and the diagnostic data comprises data regarding the size of an organ in the body part.

36. The method of claim 27, wherein the medium comprises a body part including bone, and the diagnostic data comprises data regarding bone density.

37. The method of claim 27, wherein the medium comprises a body part including an eye, and the diagnostic data comprises data regarding at least one of ocular pressure, eye geometry, or eye anomalies.

38. The method of claim 27, wherein the medium comprises a body part, and the diagnostic data comprises data regarding a foreign object in the body part.

39. The method of claim 27, wherein the standard handheld cellular telephone is configured to be capable of performing operations further including:
providing instructions regarding how to position the standard handheld cellular telephone at a location adjacent to the medium;
performing operations of claim 27 providing instructions regarding how to reposition the standard handheld cellular telephone to a new location adjacent to the medium; and
repeating the operations of claim 27

40. A method performed by a standard handheld cellular telephone having a processor, a cellular transceiver, an acoustic transmitter, an acoustic receiver, and memory for storing instructions for execution by the processor, the method comprising:
transmitting with the acoustic transmitter a first acoustic signal through a first lung;
receiving with the acoustic receiver one or more first signal propagations of the transmitted first acoustic signal after the transmitted first acoustic signal has passed at least in part through the first lung;
generating a first spectrogram from the received one or more first signal propagations;
transmitting with the acoustic transmitter a second acoustic signal through a second lung;
receiving with the acoustic receiver one or more second signal propagations of the transmitted second acoustic signal after the transmitted second acoustic signal has passed at least in part through the second lung;
generating a second spectrogram from the received one or more second signal propagations; and
comparing the first spectrogram with the second spectrogram to identify a change in a resonance frequency from the first lung and the second lung.
